(19) United States
(12) Patent Application Publication
(10) Pub. No.: US 2021/0214774 A1
(43) Pub. Date: Jul. 15, 2021

(54) METHOD FOR THE IDENTIFICATION OF ORGANISMS FROM SEQUENCING DATA FROM MICROBIAL GENOME COMPARISONS

(71) Applicant: KONINKLIJKE PHILIPS N.V., EINDHOVEN (NL)

(72) Inventors: Andrew G. Hoss, Cambridge, MA (US); Hareesh Chamarthi, Cambridge, MA (US)

(21) Appl. No.: 15/734,043

(22) PCT Filed: Aug. 13, 2019
(86) PCT No.: PCT/EP2019/071652

§ 371 Date: Dec. 1, 2020

Related U.S. Application Data
(60) Provisional application No. 62/720,198, filed on Aug. 21, 2018.

(51) Int. Cl. C12Q 1/689; G16B 30/10

(52) U.S. Cl. CPC C12Q 1/689; G16B 30/10

(57) ABSTRACT

A method (100) for characterizing a sample using a sample characterization system (400), comprising: (i) obtaining (120) sequencing data from the sample; (ii) identifying (130) a genotype of an organism in the sample by comparing the sequencing data to a set of genetic features, comprising genetic features for each of a plurality of different organisms; (iii) selecting (140) which of a plurality of reference genome sets to compare the sequencing data to; (iv) comparing (150) the sequencing data to the selected set of reference genomes; (v) identifying (160) with which reference genome in the selected set of reference genomes the sequencing data most closely aligns, the identification comprising an identification of a species or substrain; and (vi) reporting (170) one or more of the identified genotype of the organism in the sample and the identification of the species or substrain of the organism in the sample.

### FIELD OF THE DISCLOSURE

The present disclosure is directed generally to methods and systems for characterizing microorganisms in a sample.

### BACKGROUND

Traditionally, when a patient present signs of an infection, clinicians request lab tests to confirm whether the patient indeed has an infection, to classify the pathogen, and to identify with which antibiotic drugs the infection should be treated. Biological samples such as sputum, wound swabs, or blood are collected and sent to the lab for testing. Samples are spread onto nutrient-rich agar plates and left in an environment permissive for growth. If bacteria are present, then punctate colonies will form. A single colony is selected from the plate for further classification, such as for staining and/or microscopic examination to identify a species based on morphological features, and for antibiotic sensitivity testing such as identifying growth across a titrated concentration of various antibiotics.

However, due to a wide variety of factors including variability in assay procedures, differences in the colonies, contamination due to multiple organisms in a single colony, inconclusive biochemical results, and/or simply human error, microbiology results intermittently report incorrect species or inaccurate antibiotic sensitivity profiles. Additionally, depending on the hospital and their procedures, organisms are only classified down to the genus or complex level. For example, genera such as coagulase-negative Staphylococci and complexes such as Acinetobacter baumannii complex and Enterobacter cloacae complex commonly occur, but lab tests to provide resolution to the species level are commonly not performed.

Genomic analysis can help identify pathogens, and is increasingly being applied in clinical settings. For example, metagenomics software can identify the distribution of organisms within a sample by analyzing DNA sequencing. However, these tools are designed to utilize data from complex samples comprising DNA sequences from multiple organisms found in samples such as stool or saliva, rather than from samples comprising just one or a small number of different organisms. Further, these tools are not designed to verify the identity of a single organism using DNA sequencing data, and perform poorly when resolving samples down to the strain or substrain level, or when resolving a complex such as the Enterobacter cloacae complex down to the species level. Further, these tools are not designed to select or pick a suitable reference genome for genetic epidemiological investigations.

### SUMMARY OF THE DISCLOSURE

There is a continued need for methods and systems that enable the quick and accurate identification of the species or substrain of organisms in a sample comprising only one or a small number of organisms.

The present disclosure is directed to inventive methods and systems for characterizing one or more microorganisms in a sample. Various embodiments and implementations herein are directed to a system that obtains sequencing data from a microorganism in the sample. The sequencing data is compared to a set of extracted and stored genetic features which contain genetic features for a variety of different microorganisms. The genotype of the microorganism is identified and classified by the comparison, which identifies a broad category type for the microorganism. Using the identification, a set of reference genomes specific to the microorganism is selected and the sequencing data is aligned to the reference genomes in the set. Based on which reference genome in the selected set the sequencing data most closely aligns with, the system identifies a specific species or substrain of the microorganism. The system reports the classification of the genotype of the microorganism and the identification of the specific species or substrain of the microorganism. A treatment plan specific to the identified species or substrain is selected and implemented, which may include a selection of a specific antibiotic or other treatment agent.

Generally in one aspect, is a method for characterizing a sample comprising one or more different organisms. The method includes: (i) obtaining the sample from a culture; (ii) obtaining sequencing data from the one or more different organisms in the sample; (iii) identifying a genotype of an organism in the sample by comparing the sequencing data to a set of genetic features, the set of genetic features comprising genetic features for each of a plurality of different organisms, the genetic features for each of the plurality of different microorganisms generated by: extracting, from a plurality of sequenced genomes representing two or more varieties of an organism, one or more genetic features unique to the organism; normalizing the one or more extracted genetic features; and storing the normalized set of genetic features in memory; (iv) selecting which of a plurality of reference genome sets to compare the sequencing data to, each of the plurality of reference genome sets comprising a plurality of diverse reference genomes for an organism; (v) comparing the sequencing data to the selected set of reference genomes; (vi) identifying, based on the comparison, with which reference genome in the selected set of reference genomes the sequencing data most closely aligns, the identification further comprising an identification of a species or substrain of the organism in the sample; and (vii) reporting, via a user interface, one or more of the identified genotype of the organism in the sample and the identification of the species or substrain of the organism in the sample.

According to an embodiment, the method further includes implementing, based on the report, a treatment plan specific to the identified species or substrain of the organism. According to an embodiment, the treatment plan comprises an antibiotic or other agent selected based on the identified species or substrain of the organism. According to an embodiment, the identified species or substrain of the organism is used for an epidemiological plan or study.

According to an embodiment, each of the plurality of reference genome sets is generated by: (i) selecting a plurality of reference genomes for an organism; (ii) identifying, using the set of genetic features for that organism, the sequence similarity of each of the selected plurality of reference genomes; (iii) curating the plurality of reference genomes based on the identified sequence similarity; and (iv) storing the curated reference genome set in memory. According to an embodiment, curation comprises eliminating from the reference genome set a reference genome that has a sequence similarity relative to another reference genome in the selected plurality of reference genomes above a predetermined threshold.

According to an embodiment, the method further includes determining, based on the identified genotype of the organism, that the sequencing data comprises sequences from two or more organisms; and separating the sequencing data from each of the two or more organisms in the sample into a separate sequencing data file for each organism.

According to an embodiment, comparing the sequencing data to the selected set of reference genomes comprises alignment of the sequencing data with each of the reference genomes in the set.

According to an embodiment, identifying with which reference genome in the selected set of reference genomes the sequencing data most closely aligns comprises an analysis of sequence similarity and/or a phylogenetic analysis.

According to an embodiment, the identification of the species or substrain of the organism in the report comprises a quantitative measure of the identification.

According to an embodiment, the one or more genetic features comprise one or more genetic loci and/or one or more k-mers.

According to another aspect is a system configured to characterize a sample comprising one or more different organisms. The system includes: sequencing data obtained from a cultured sample; a data structure configured to store a set of genetic features comprising genetic features for each of a plurality of different organisms, and a plurality of reference genome sets each set comprising a plurality of diverse reference genomes for an organism; a processor configured to: (i) identify a genotype of an organism in the sample by comparing the sequencing data to the set of genetic features, the set of genetic features comprising genetic features for each of a plurality of different organisms; (ii) select to which of the plurality of reference genome sets to compare the sequencing data; (iii) compare the sequencing data to the selected set of reference genomes; (iv) identify, based on the comparison, with which reference genome in the selected set of reference genomes the sequencing data most closely aligns, the identification further comprising an identification of a species or substrain of the organism in the sample; and a user interface configured to report of one or more of the identified genotype of the organism in the sample and the identification of the species or substrain of the organism in the sample.

According to an embodiment, the processor is further configured to generate a genetic feature set by: (i) extracting, from a plurality of sequenced genomes representing two or more varieties of an organism, one or more genetic features unique to the organism; (ii) normalizing the one or more extracted genetic features; and (iii) storing the normalized set of genetic features in memory.

According to an embodiment, the processor is further configured to generate each of the plurality of reference genome sets by: (i) selecting a plurality of reference genomes for an organism; (ii) identifying, using the set of genetic features for that organism, the sequence similarity of each of the selected plurality of reference genomes; (iii) curating the plurality of reference genomes based on the identified sequence similarity; and (iv) storing the curated reference genome set in memory.

According to an embodiment, the processor is further configured to: determine, based on the identified genotype of the organism, that the sequencing data comprises sequences from two or more organisms; and separate the sequencing data from each of the two or more organisms in the sample into a separate sequencing data file for each organism.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for characterizing a sample comprising one or more different microorganisms, in accordance with an embodiment.

FIG. 2 is a flowchart of a method for generating a set of genetic features, in accordance with an embodiment.

FIG. 3 is a flowchart of a method for generating a set of reference genomes, in accordance with an embodiment.

FIG. 4 is a schematic representation of a system for characterizing a sample comprising one or more different microorganisms, in accordance with an embodiment.

### DETAILED DESCRIPTION OF EMBODIMENTS

The present disclosure describes various embodiments of a system and method for characterizing one or more microorganisms in a sample. More generally, Applicant has recognized and appreciated that it would be beneficial to provide a system configured to identify the species and/or substrain of a microorganism in a sample. The system, which may optionally comprise a sequencing platform, generates or receives sequencing data, such as whole genome data and/or genome assemblies, from a sample believed to comprise an infectious pathogen. The system compares the sequencing data to a set of genetic features for a variety of different microorganisms, and the genotype of the microorganism is identified and classified. Using this identification, a set of reference genomes specific to the microorganism is selected and the sequencing data is aligned to the reference genomes in the selected set. Based on which reference genome the sequencing data most closely aligns with, the system identifies a specific species or substrain of the microorganism. The system reports the classification of the genotype of the microorganism and the identification of the specific species or substrain. A treatment plan specific to the identified species or substrain is selected and implemented, which may include a selection of a specific antibiotic or other treatment agent.

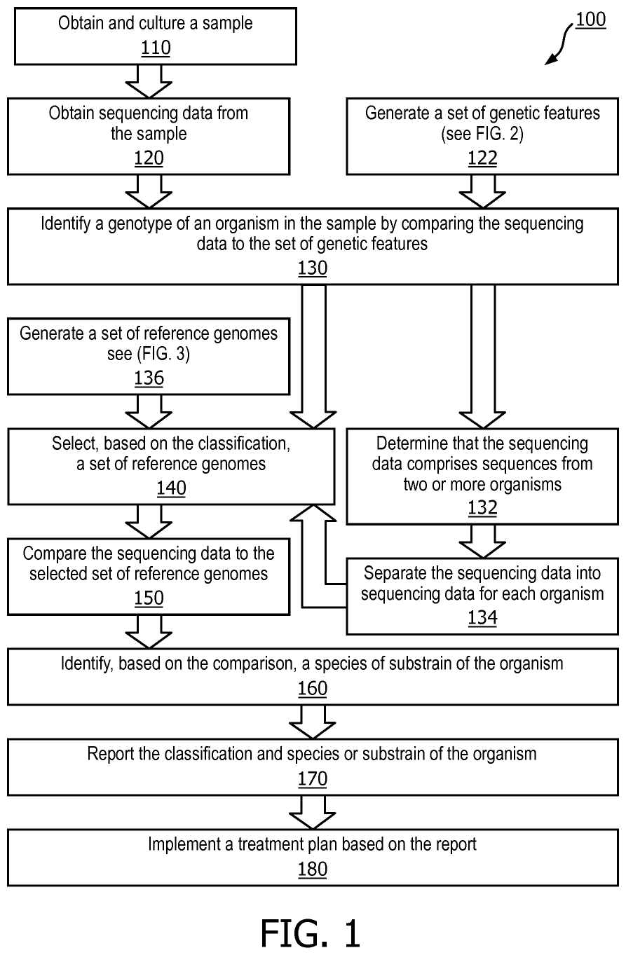

Referring to FIG. 1, in one embodiment, is a flowchart of a method 100 for characterizing a sample comprising one or more different microorganisms using a sample characterization system. The sample characterization system may be any of the systems described or otherwise envisioned herein, and may comprise any of the components described or otherwise envisioned herein.

At step 110 of the method, a sample is obtained and cultured. The sample may be any sample containing or suspected of containing a microorganism of interest. For example, the sample may contain a pathogenic microorganism such as an infectious bacteria species. The sample may contain a single microorganism or multiple different microorganisms, which may be different genera, species, and/or substrains, among other possibilities. The sample may be obtained from a surface, such as swabs of surfaces, tools, equipment, or other surfaces, including in a hospital setting. The sample may be obtained from an individual or animal, such as from a wound or infectious location including skin, blood, urine, cerebrospinal fluid, lymph fluid, and/or any location. The sample may be obtained using any method for capturing a sample, including a swab, fluid collection, and/or any other method. Once collected, the sample is cultured on a solid, semi-solid, or liquid medium. Any method for culturing a sample may be utilized. The sample may be cultured as long as necessary for sufficient growth for analysis.

According to one embodiment, the sample is collected from a patient in a hospital or other healthcare setting upon visual inspection or lab test results suggesting the presence of an infection or pathogen. A healthcare specialist requests an analysis of the infection in order to identify the pathogen, which will inform or select a treatment plan for the infection. A sample is collected, cultured, and analyzed as described or otherwise envisioned herein in order to identify the responsible pathogen(s) and determine a treatment plan which the healthcare professional then implements.

It is recognized that there is no limitation to the source of the sample. For example, the sample may be collected from a single location or several locations. According to an embodiment, samples may be obtained over a plurality of time points. For example, the sample may be collected from one or more than one location over two or more points in time. The two or more points in time may be selected based on a wide variety of different criteria and/or methodologies. As another embodiment, the samples are collected from a single location, several locations, or many locations over two or more points in time.

At step 120 of the method, the sample characterization system generates sequencing data from the sample, or otherwise receives sequencing data obtained from the sample. The sequencing data may be DNA sequencing data, and may be obtained using any method for obtaining sequencing data. According to an embodiment, cells from the cultured sample are isolated or obtained from the culture and are prepared for DNA sequencing. DNA is extracted from the cells and used to generate sequencing data. Methods for preparing the cells, extracting and preparing the DNA, and generating the sequencing data may be any such methods known in the art. The output of step 120 may be, for example, sequencing read files in a format such as FASTA, FASTQ, and/or any other file format. The generated and/or received sequencing data may be comprise a plurality of sequence reads or read fragments obtained from the sample.

The sequencing data may be utilized immediately for analysis as described or otherwise envisioned herein, and/or the sequencing data may be stored for later analysis. For example, the obtained sequencing data may be fed directly into the sample characterization system for analysis, or may be stored locally or remotely, within or separate from the sample characterization system, for later analysis. The sequencing data may exist in an unassembled, partially assembled, or complete genome format.

According to an embodiment, the sample characterization system comprises a sequencing platform configured to obtain sequencing data from the sample. The sequencing platform can be any sequencing platform, including but not limited to any systems described or otherwise envisioned herein. For example, the sequencing platform can be a real-time single-molecule sequencing platform, such as a pore-based sequencing platform, although many other sequencing platforms are possible. The sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner.

According to an embodiment, the sample characterization system receives the sequencing data obtained by a sequencing platform from the sample. For example, the sample characterization system may be in communication or otherwise receive the sequencing data from a local or remote sequencing platform which is separate from the sample characterization system.

The generated and/or received sequencing data may be stored in a local or remote database for use by the sample characterization system. For example, the sample characterization system may comprise a database to store the sequencing data, and/or may be in communication with a database storing the sequencing data. These databases may be located with the sample characterization system or may be located remote from the sample characterization system, such as in cloud storage and/or other remote storage.

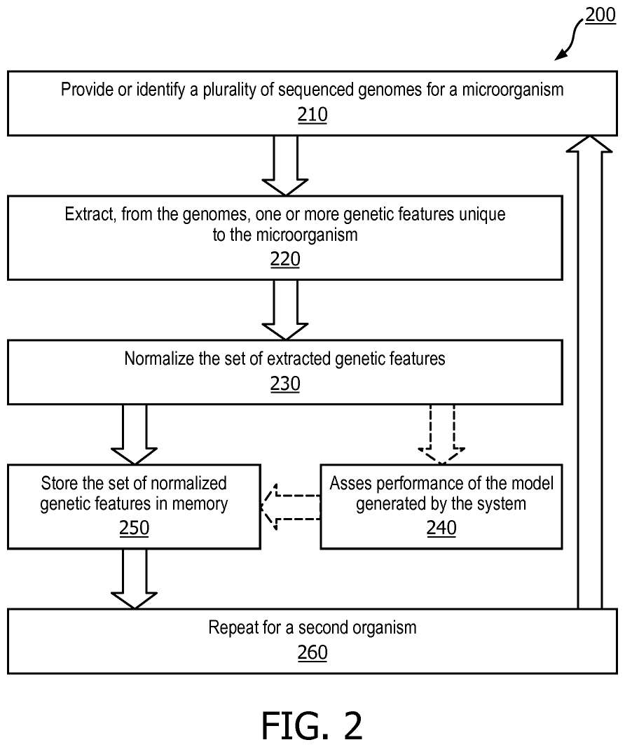

At step 122 of the method, which may be performed at a time separate from one or more other steps of the method, a set of genetic features for an organism are generated. Referring to FIG. 2, in one embodiment, is a flowchart of a method 200 for generating a set of genetic features for one or more organisms.

At step 210 of the method, a plurality of sequenced and assembled genomes from an organism are generated or received by the sample characterization system. The plurality of sequenced genomes may be obtained using any method for sequencing DNA. The genomes may be stored locally within or remotely from the sample characterization system. According to an embodiment, the sample characterization system generates and/or receives a plurality of sequenced genomes for each of a plurality of different organisms. The sample characterization system can thus generate distinguishing genetic features for each of the plurality of different organisms.

According to an embodiment, a user defines via a user interface or programming the level of classification needed by the sample characterization system. Among other options, a user may determine that only high-level classification is required, or may determine that species-level classification is required, or may determine that substrain classification is required. For example, for Enterobacter cloacae complex, the system may be directed to distinguish Enterobacter from the complex level down to the species level.

According to an embodiment, based on the level of classification defined or selected by the user, a set of sequenced organisms with assembled genomes spanning the classification level are required. For example, for Enterobacter cloacae complex, a set of genomes comprising all species within the complex would be used. The number of samples in this set may be greater than or equal in sample size to genome reference set curated in subsequent step. Accordingly, the user's selected level of classification may be used to identify which and how many genomes to utilize in this method. The plurality of sequenced and assembled genomes may be obtained by the system via sequencing, and/or may be obtained by the system from a source of genomes such as public and/or private sequencing databases. For example, sequenced genomes may be obtained from a reference database such as NCBI RefSeq, among many others.

At step 220 of the method, the sample characterization system extracts one or more genetic features from the set of sequenced and assembled genomes for an organism. According to an embodiment, the one or more extracted genetic features are unique to that organism, which allows the system to discern between organisms in subsequent steps of the process as described or otherwise envisioned herein. The genetic features can be identified using any method for identifying and extracting genetic features from a set of sequenced and assembled genomes, including using an algorithm or software to identify and extract features. For example, the genetic features may be one or more k-mers and/or genes that best define the organism in question. Accordingly, the genetic features may be any single gene, a set of genes, or sets of loci, among other options. According to an embodiment, each genome in the set of sequenced and assembled genomes for an organism are processed using the same genetic feature(s).

According to an embodiment, the sample characterization system identifies a set of candidate genetic features. The system then analyzes the identified set of candidate genetic features to identify only stable, or the most stable, genetic features. Features that do not pass criteria, for example, can be removed from the set or otherwise eliminated from consideration as a final genetic feature. For example, a region or gene which is not present across a majority of the genomes with the set, or has a low sequence conservation of coding and/or noncoding regions, or has a low concordance of gene annotations from sequence using a CDS prediction and/or protein homolog, or is under selective pressure, or does not pass a detection threshold as defined by user, among other options, can be removed from the set or otherwise eliminated from consideration as a final genetic feature. The output of this step, therefore, will be a set of one or more stable final genetic features.

At step 230 of the method, the set of final genetic features is normalized. For example, the genetic features can be normalized across the set of sequenced and assembled genomes for an organism. For example, the abundance of genetic features can be normalized based on the total amount of sequencing reads are observed for an individual sample. For example, if using a gene-based approach, multi-sequence alignment can be used within each genetic feature, including identifying an optimal sequence alignment of the set of genetic features. If using k-mers, the genetic features can be normalized based on k-mer frequency and/or based on other parameters. For example, if a gene or region is sequenced at a greater depth based on the k-mer frequency, the system can normalize the reads in the file or perform similar normalizing. Other normalization methods including normalizing using gene length, conservation scores, divergent time estimations, or adjusting the depth of sequencing data, among others.

At step 240 of the method, which may be optional, the system may review or assess the performance of the set of normalized final genetic features. According to an embodiment, the system may utilize a phylogenetic analysis to assess the discriminatory power of the genetic features, based on the user-selected classification level. For example, the system may examine the ability of the normalized final genetic features to identify or classify an organism, and/or to distinguish between two or more organism, which may optionally be based on a threshold, a similarity, or other comparison mechanism. According to an embodiment, the system may utilize the results of the assessment to improve the discriminatory power of the model by including more genetic features such as polymorphic genes/regions, and/or by reducing highly conserved genes/regions if needed to improve discriminatory ability of the model.

The output of step 230 and/or 240 is a set of normalized final genetic features. The set of normalized final genetic features may be in any format utilizable by the sample characterization system. For example, the set may be or comprise a list of genetic sequences with informative genetic features.

At step 250 of the method, the set of normalized final genetic features are stored in a local or remote database for retrieval and use by the sample characterization system. For example, the sample characterization system may comprise a database to store the genetic features, and/or may be in communication with a database storing the genetic features. These databases may be located with the sample characterization system or may be located remote from the sample characterization system, such as in cloud storage and/or other remote storage.

At step 260 of the method, the process for creating a set of normalized final genetic features can be repeated for one or more additional organisms.

Returning to FIG. 2, at step 130 of the method, in accordance with an embodiment, the sample characterization system identifies a genotype of a microorganism in the sample by comparing the sequencing data to the set of genetic features, which comprises genetic features for each of a plurality of different organisms. For example, the system may align k-mers in the sequencing data with the k-mers, genes, and/or genetic regions found within the set of genetic features, although many other methods of comparison are possible. The organism in the sample from which the sequencing data was obtained may be identified or characterized based on sufficient similarity, shown by the alignment, between the sequencing data and a set of genetic features for a particular organism. For example, the comparison of the sequencing data to the set of genetic features may determine that the organism in the sample from which the sequencing data was obtained is most likely Enterobacter, or most likely Staphylococci, among many other organisms. This determination may include a probability, ranking, or other quantitative assessment or measurement. As just one example, phylogenetic software may be used or adapted to perform the comparison and identify a likely organism.

At optional step 132 of the method, the system determines that the sequencing data comprises data from two or more organisms. This determination may be based on the comparison of the sequencing data to the set of genetic features in step 130 of the method. For example, the sequencing data may align with the genetic features of two or more organisms, thus indicating the presence of sequencing data from two or more organisms. This may involve a threshold to filter out spurious or minor results, and thus the method may include comparison of the results to a filtering threshold. For example, if the system determines that only a minor portion of the sequencing data is from a subsequent organism, which may be below a certain percentage, then the system may ignore that sequencing data and/or the identification of the second organism based on the low-level sequencing data. As another example, if the system determines that a substantial portion of the sequencing data is from each of two or more organisms, such as 45% from a first organism and 55% from a second organism, the system may determine from comparison to the threshold that there are two or more organisms present in the sample and will not ignore or remove any sequencing data or an identified genotype of any of the organisms. Thus, the threshold may be set at any level necessary for discrimination of non-minor percentages. This threshold may be determined by a user, a setting of the system, and/or based on any other parameter.

At optional step 134 of the method, the system separates the sequencing data into a separate file for each of the two or more identified organisms. This may be performed, for example, by aligning k-mers in the sequencing data with reference genomes for each of the identified organisms, or by any other method. Once the sequencing data is separated into files for each identified organism, each set of sequencing data can proceed independently through subsequent steps of the method. The reporting step, discussed herein, may then comprise a report relevant to and describing each of the two or more identified organisms.

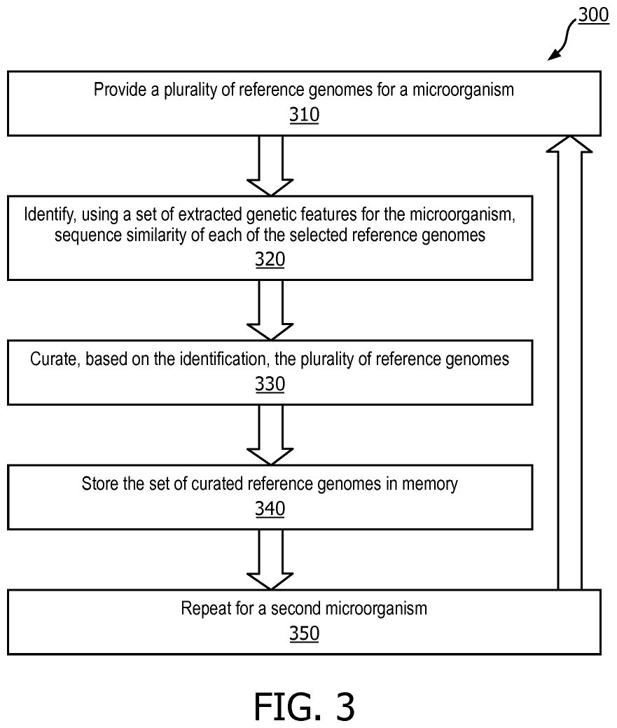

At step 136 of the method, which may be performed at a time separate from one or more other steps of the method, a set of reference genomes for an organism are generated. Referring to FIG. 3, in one embodiment, is a flowchart of a method 300 for generating a set of reference genomes for one or more organisms.

At step 310 of the method, a plurality of reference genomes for each of a plurality of organisms is provided. Preferably, each plurality of reference genomes comprises or otherwise represents a variety of different variations of the organism, such as different substrains of the organism. According to another embodiment, the variations may represent different species or other classifications of the organism. For example, reference genomes for a plurality of Staphylococci species and/or substrains may be provided to enable the creation of a suitable set of reference genomes for Staphylococci analysis. The reference genomes may be generated by the system and/or may be obtained from remote or local public and/or private databases. For example the reference genomes may be NCBI RefSeq reference genomes, or any other genomes. These pre-analysis reference genomes may be stored in a local or remote database for use by the system in method 300.

At step 320 of the method, each of the genomes in a plurality of reference genomes for an organism is compared to every other genome in order to determine sequence similarity of the plurality of genomes. This may be performed by any software, algorithm, or alignment method capable of aligning or otherwise comparing two or more genomes. The output of step 320 may be a report of the sequence similarity of the plurality of genomes in reference to each other.

At step 330 of the method, one or more genomes in the plurality of reference genomes for an organism may be curated. This may be performed, for example, to ensure a comprehensive set of diverse reference genomes without substantial repetition or other undesired characteristics. According to an embodiment, curation comprises eliminating any duplicate reference genomes from within the plurality of reference genomes. This may comprise, for example, eliminating a reference genome that has a sequence similarity relative to another reference genome in the selected plurality of reference genomes above a predetermined threshold. Other options for curation of the plurality of reference genomes are possible. For example, the analysis may determine that the reference genomes in the set are too similar, and thus that additional reference genomes are necessary. Curation may comprise, therefore, identification of additional reference genomes for the organism, either known to comprise additional diversity or expected to comprise additional diversity, and the analysis can be performed again.

The output is a set of diverse reference genomes for an organism. The set of diverse reference genomes may be in any format utilizable by the sample characterization system. The set of diverse reference genomes may comprise 1 reference genome or many reference genomes, depending on the known or expected diversity of the organism, of the sample, and/or based on a setting by the user or the system.

At step 340 of the method, the set of reference genomes for the organism are stored in a local or remote database for retrieval and use by the sample characterization system. For example, the sample characterization system may comprise a database to store the set of reference genomes, and/or may be in communication with a database storing the set of reference genomes. These databases may be located with the sample characterization system or may be located remote from the sample characterization system, such as in cloud storage and/or other remote storage.

At step 350 of the method, the process for creating a set of reference genomes can be repeated for one or more additional organisms.

Returning to method 100 in FIG. 2, at step 140 of the method, in accordance with an embodiment, a set of reference genomes is selected. According to an embodiment, the set of reference genomes is selected by the sample characterization system based on the identified genotype of the organism. The set of reference genomes is generated, according to an embodiment, via the method described in conjunction with FIG. 3. For example, if the genotype characterization in a previous step identifies Staphylococci as being present in the sample and the primary or only source of the sequencing data, the system will call the set of Staphylococci reference genomes stored in memory.

According to another embodiment, at step 140 of the method the set of reference genomes is selected or determined by a user. For example, a user may know that a sample is an Enterobacter and selects the set of Enterobacter reference genomes stored in memory for the analysis. This may be, for example, to determine which Enterobacter species are present in the sample. The user may make the selection or determination using a user interface of the sample characterization system, among other methods.

At step 150 of the method, in accordance with an embodiment, the sample characterization system compares the sequencing data to the selected set of reference genomes. To continue the example, if the genotype characterization in a previous step identifies Staphylococci as being present in the sample and the primary or only source of the sequencing data, the system will call the set of Staphylococci reference genomes stored in memory, and will compare the sequencing data to the set of Staphylococci reference genomes retrieved from memory. The comparison may be performed using any method for alignment or comparison of sequencing data with a reference genome. According to an embodiment, the system identifies one or more reference genome in the selected set with which the sequencing data, and thus the organism in the sample, most closely align.

According to an embodiment, the system utilizes an algorithm or software, such as a metagenomics tool, to compare some or all of the sequencing data to the selected set of reference genomes, such as trying to align the sequencing data with each of the reference genomes in the selected set. For example, the system may use software such as or similar to BLAST to identify the highest sequence similarity between the sequencing data and one of the reference genomes for a single gene or locus or for multiple genes or loci. According to an embodiment, the system may use phylogenetic software to cluster the sequencing data and/or reference genomes in order to identify the reference genome most similar to the sequencing data and thus most similar to the organism in the sample.

At step 160 of the method, the system identifies the most likely species, substrain, or other classification of the organism in the sample from which the sequencing data was obtained, based on the comparison in the previous step. For example, the system may identify a single species or substrain for an organism based on the most similarity between the sequencing data and the reference genome for that identified single species or substrain. This identification may comprise a likelihood, confidence, percent similarity, or other quantification. As just one example, the system may determine that the sequencing data comprises 97.3% similarity to substrain E of a particular species of an organism based on the comparison between the reference genomes for that organism and the sequencing data. The system may simultaneously determine a 90.5% similarity to substrain F of the species, and will report the similarity to substrain E in a subsequent step, since it is a higher similarity score. Alternatively, the system may report similarity or alignment scores for some or all of the reference genomes in the selected set of reference genomes, and may rank the scores with, for example, the highest scores listed first.

At step 170 of the method, the system reports one or more of the identified genotype of the organism in the sample and/or the identification of the species, substrain, or other classification of the organism in the sample. The report may be provided via a user interface of the system, which can be any device or system that allows information to be conveyed and/or received, and may include a display, a mouse, and/or a keyboard for receiving user commands. The report may be a visual display, a printed text, an email, an audible report, a transmission, and/or any other method of conveying this information. The report may be provided locally or remotely, and thus the system or user interface may comprise or otherwise be connected to a communications system. For example, the system may communicate a report over a communications system such as the internet or other network.

According to an embodiment, the report may comprise the determined sequence similarity from a previous step of the method as described or otherwise envisioned herein. In the case of multiple organisms represented in the sequencing data, as described herein, the report may comprise information about each of these multiple organisms.

At step 180 of the method, in one embodiment, the information provided by the report is utilized for or as part of an epidemiological study, investigation, or other analysis. For example, the information could be utilized to examine the distribution and/or determinant(s) of an infection, or the possibility of an infection or disease. The information could also or alternatively be utilized to examine the prevention, treatment, or control of infection or disease.

According to another embodiment, at step 180 of the method a treatment plan is generated and/or implemented based on the information in the report. This may be an automated or a manual process. For example, a healthcare professional may receive or otherwise process the report and generate a treatment plan. Alternatively, the system may automatically generate a treatment plan based on the identification of the one or more organisms in the sample. For example, the system may be programmed to identify which of a plurality of possible treatment plans should be generated, recalled from memory, or otherwise identified based on which organism is in the sample. Thus, the treatment plan may be specific to the species, substrain, or other classification of organism in the sample as identified by the sample characterization system. The healthcare professional may then utilize the generated treatment plan to implement treatment or care of the surface, individual, animal, or other source of the sample from which the species, substrain, or other classification of organism was made. As just one example, the treatment plan may comprise a specific antibiotic or other agent used to treat the presence of or infection with the specific species, substrain, or other classification of organism found in the sample.

According to yet another embodiment, at step 180 of the method the information provided by the report is utilized to compare to a finding of a species, substrain, or other classification of organism identified by another analytical process. For example, any one of a number of possible microbiological methods may be utilized to identify an organism in a sample, and that identification can be compared to the information in the report. The information may match, where the identification is exactly the same or the method described herein provides a more specific identification of a particular species or substrain compared to the identification made by the other microbiological method. Alternatively, the two identifications may not match, in which case the report provided by the method described herein may be preferentially utilized, the analysis may be repeated with the same sample or a new sample, and/or a subsequent analytical method may be utilized. Many other options are possible.

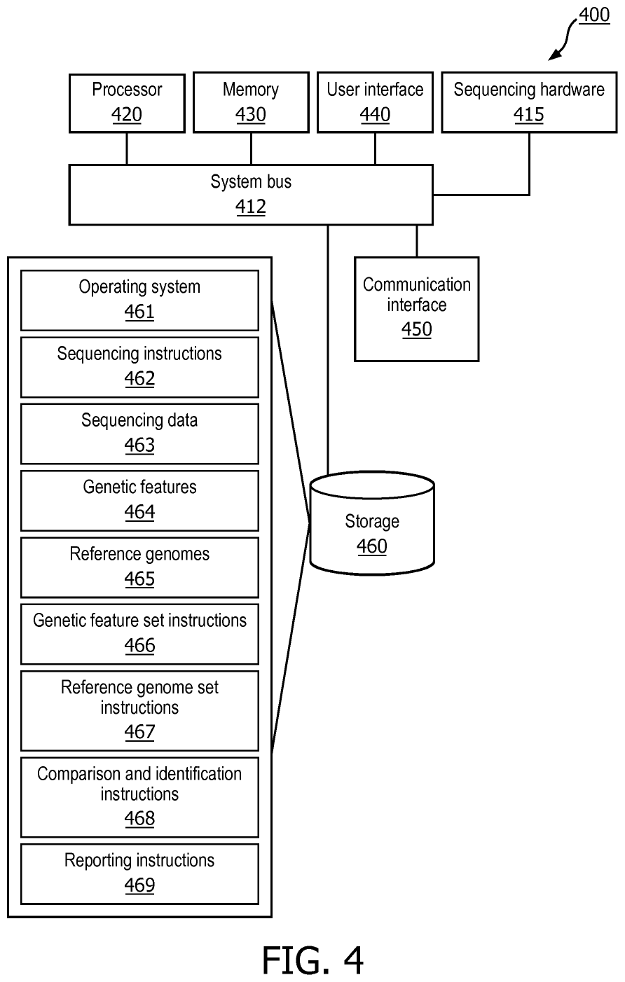

Referring to FIG. 4, in one embodiment, is a schematic representation of a sample characterization system 400 for generating a genome reference. System 400 may be any of the systems described or otherwise envisioned herein, and may comprise any of the components described or otherwise envisioned herein.

According to an embodiment, system 400 comprises one or more of a processor 420, memory 430, user interface 440, communications interface 450, and storage 460, interconnected via one or more system buses 412. In some embodiments, such as those where the system comprises or directly implements a sequencer or sequencing platform, the hardware may include additional sequencing hardware 415 such as a real-time single-molecule sequencer, including but not limited to a pore-based sequencer, although many other sequencing platforms are possible. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 430 or storage 460 or otherwise processing data to, for example, perform one or more steps of the method. Processor 420 may be formed of one or multiple modules. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 430 can take any suitable form, including a non-volatile memory and/or RAM. The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user. The user interface can be any device or system that allows information to be conveyed and/or received, and may include a display, a mouse, and/or a keyboard for receiving user commands In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (NIC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

Storage 460 may include one or more machine-readable storage media such as read-only memory (ROM), random-access memory (RAM), magnetic disk storage media, optical storage media, flash-memory devices, or similar storage media. In various embodiments, storage 460 may store instructions for execution by processor 420 or data upon which processor 420 may operate. For example, storage 460 may store an operating system 461 for controlling various operations of system 400. Where system 400 implements a sequencer and includes sequencing hardware 415, storage 460 may include sequencing instructions 462 for operating the sequencing hardware 415, and sequencing data 463 obtained by the sequencing hardware 415. Storage 460 may also store a set of genetic features 464 and a set of reference genomes 465.

It will be apparent that various information described as stored in storage 460 may be additionally or alternatively stored in memory 430. In this respect, memory 430 may also be considered to constitute a storage device and storage 460 may be considered a memory. Various other arrangements will be apparent. Further, memory 430 and storage 460 may both be considered to be non-transitory machine-readable media. As used herein, the term non-transitory will be understood to exclude transitory signals but to include all forms of storage, including both volatile and non-volatile memories.

While sample characterization system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where one or more components of system 400 is implemented in a cloud computing system, the various hardware components may belong to separate physical systems. For example, processor 420 may include a first processor in a first server and a second processor in a second server. Many other variations and configurations are possible.

According to an embodiment, storage 460 of sample characterization system 400 may store one or more algorithms and/or instructions to carry out one or more functions or steps of the methods described or otherwise envisioned herein. For example, processor 420 may comprise, among other instructions, genetic feature set instructions 466, reference genome set instructions 467, comparison and identification instructions 368, and/or reporting instructions 369.

According to an embodiment, genetic feature set instructions 466 direct the system to generate a set of genetic features for each of a plurality of different organisms. For example, according to an embodiment, the sample characterization system generates or receives a plurality of sequenced and assembled genomes for an organism, and extracts one or more genetic features from the plurality of genomes. The one or more extracted genetic features may be unique to that organism, and may be identified using any method for identifying and extracting genetic features from a set of sequenced and assembled genomes. The extracted genetic features may be any single gene, a set of genes, or sets of loci, among other options. The genetic feature set instructions 466 direct the system normalize the genetic features across the set of sequenced and assembled genomes for an organism. Many methods for normalization are possible, as described or otherwise envisioned herein. The system may optionally review or assess the performance of the set of normalized final genetic features as described or otherwise envisioned herein. According to another embodiment, the set of genetic features may be otherwise processed or analyzed. For example, the set of genetic features may be curated, filtered or refined using a variety of different possible approaches or methods.

The instructions may direct the system to store the normalized final genetic features may then be stored in a local or remote database for retrieval and use by the sample characterization system. The database may be located with the sample characterization system or may be located remote from the sample characterization system, such as in cloud storage and/or other remote storage.

According to an embodiment, reference genome set instructions 467 direct the system to generate a set of reference genomes for each of a plurality of organisms. For example, according to an embodiment, the sample characterization system obtains a plurality of reference genomes for each of the plurality of organisms. Preferably, each plurality of reference genomes comprises or otherwise represents a variety of different variations of the organism, such as different species or substrains of the organism. The reference genomes may be generated by the system and/or may be obtained from remote or local public and/or private databases.

The reference genome set instructions 467 direct the system to compare each of the plurality of reference genomes to every other genome in order to determine sequence similarity of the plurality of genomes. This may be performed by any software, algorithm, or alignment method capable of aligning or otherwise comparing two or more genomes. The system may then curate one or more of the plurality of reference genomes based on the results of the comparison in order to ensure a comprehensive set of diverse reference genomes without substantial repetition or other undesired characteristics. For example, the system may remove any duplicate reference genomes from within the plurality of reference genomes. This may comprise eliminating a reference genome that has a sequence similarity relative to another reference genome in the selected plurality of reference genomes above a predetermined threshold. Many other options for curation of the plurality of reference genomes are possible.

The instructions may direct the system to store the curated set of reference genomes in a local or remote database for retrieval and use by the sample characterization system. The database may be located with the sample characterization system or may be located remote from the sample characterization system, such as in cloud storage and/or other remote storage.

According to an embodiment, comparison and identification instructions 368 direct the system to identify a genotype of a microorganism in the sample by comparing the sequencing data to the set of genetic features, which comprises genetic features for each of a plurality of different organisms. The organism in the sample from which the sequencing data was obtained may be identified or characterized based on sufficient similarity, shown by the alignment, between the sequencing data and a set of genetic features for a particular organism. This determination may include a probability, ranking, or other quantitative assessment or measurement. As just one example, phylogenetic software may be used or adapted to perform the comparison and identify a likely organism.

Comparison and identification instructions 368 also direct the system to select a set of reference genomes based on the identified genotype of the organism. For example, if the genotype characterization in a previous step identifies Staphylococci as being present in the sample and the primary or only source of the sequencing data, the system will call the set of Staphylococci reference genomes stored in memory.

Comparison and identification instructions 368 further direct the system to compare the sequencing data to the selected set of reference genomes. The comparison may be performed using any method for alignment or comparison of sequencing data with a reference genome. According to an embodiment, the system identifies one or more reference genome in the selected set with which the sequencing data, and thus the organism in the sample, most closely align. According to an embodiment, the system utilizes an algorithm or software, such as a metagenomics tool, to compare some or all of the sequencing data to the selected set of reference genomes, such as trying to align the sequencing data with each of the reference genomes in the selected set. For example, the system may use software such as or similar to BLAST to identify the highest sequence similarity between the sequencing data and one of the reference genomes for a single gene or locus or for multiple genes or loci. According to an embodiment, the system may use phylogenetic software to cluster the sequencing data and/or reference genomes in order to identify the reference genome most similar to the sequencing data and thus most similar to the organism in the sample.

According to an embodiment, and/or reporting instructions 369 direct the system to report one or more of the identified genotype of the organism in the sample and/or the identification of the species, substrain, or other classification of the organism in the sample. For example, according to an embodiment, the sample characterization system generates a report and provides the report via a user interface or via a communications network. According to an embodiment, the report may comprise the determined sequence similarity from a previous step of the method as described or otherwise envisioned herein. In the case of multiple organisms represented in the sequencing data, as described herein, the report may comprise information about each of these multiple organisms.

The reporting instructions 369 may also direct the system to generate a treatment plan based on the identification of the one or more organisms in the sample. For example, the instructions may inform the system which of a plurality of possible treatment plans should be generated, recalled from memory, or otherwise identified based on which organism is in the sample. Thus, the treatment plan may be specific to the species, substrain, or other classification of organism in the sample as identified by the sample characterization system. As just one example, the treatment plan may comprise a specific antibiotic or other agent used to treat the presence of or infection with the specific species, substrain, or other classification of organism found in the sample.

According to an embodiment, the healthcare professional may utilize the generated treatment plan to implement treatment or care of the surface, individual, animal, or other source of the sample from which the species, substrain, or other classification of organism was made.

The sample characterization approach described or otherwise envisioned herein provides numerous advantages over existing systems. For example, the system improves the accuracy and speed with which organisms within a sample are identified. In a clinical setting in which an individual is fighting an infection, quickly and accurately identifying the pathogen(s) participating in the infection can lead to faster and more accurate treatment. This can mean the difference between life and death in many settings and/or with many infections. Using the approach and/or system described or otherwise envisioned herein, a clinician or other healthcare provider can make significantly improved and more informed decisions, and can better treat dangerous and often life-threatening infections.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of;” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing a sample comprising one or more different organisms, comprising:
obtaining the sample from a culture;
obtaining sequencing data from the one or more different organisms in the sample;
identifying a genotype of an organism in the sample by comparing the sequencing data to a set of genetic features, the set of genetic features comprising genetic features for each of a plurality of different organisms, the genetic features for each of the plurality of different microorganisms generated by: (i) extracting, from a plurality of sequenced genomes representing two or more varieties of an organism, one or more genetic features unique to the organism; (ii) normalizing the one or more extracted genetic features; and (iii) storing the normalized set of genetic features in memory;
selecting which of a plurality of reference genome sets to compare the sequencing data to, each of the plurality of reference genome sets comprising a plurality of diverse reference genomes for an organism;
comparing the sequencing data to the selected set of reference genomes;
identifying, based on the comparison, with which reference genome in the selected set of reference genomes the sequencing data most closely aligns, the identification further comprising an identification of a species or substrain of the organism in the sample; and
reporting, via a user interface, one or more of the identified genotype of the organism in the sample and the identification of the species or substrain of the organism in the sample.

2. The method of claim 1, wherein the selection of which of a plurality of reference genome sets to compare the sequencing data to is based on the genotype of the organism in the sample, or is determined by a user.

3. The method of claim 1, further comprising implementing, based on the report, a treatment plan specific to the identified species or substrain of the organism.

4. The method of claim 1, wherein each of the plurality of reference genome sets is generated by: (i) selecting a plurality of reference genomes for an organism; (ii) identifying, using the set of genetic features for that organism, the sequence similarity of each of the selected plurality of reference genomes; (iii) curating the plurality of reference genomes based on the identified sequence similarity; and (iv) storing the curated reference genome set in memory.

5. The method of claim 4, wherein curation comprises eliminating from the reference genome set a reference genome that has a sequence similarity relative to another reference genome in the selected plurality of reference genomes above a predetermined threshold.

6. The method of claim 1, further comprising:
determining, based on the identified genotype of the organism, that the sequencing data comprises sequences from two or more organisms; and
separating the sequencing data from each of the two or more organisms in the sample into a separate sequencing data file for each organism.

7. The method of claim 1, wherein comparing the sequencing data to the selected set of reference genomes comprises alignment of the sequencing data with each of the reference genomes in the set.

8. The method of claim 1, wherein identifying with which reference genome in the selected set of reference genomes the sequencing data most closely aligns comprises an analysis of sequence similarity and/or a phylogenetic analysis.

9. The method of claim 1, wherein the identification of the species or substrain of the organism in the report comprises a quantitative measure of the identification.

10. The method of claim 1, wherein the one or more genetic features comprise one or more genetic loci and/or one or more k-mers.

11. A system for characterizing a sample comprising one or more different organisms, comprising:
sequencing data obtained from a cultured sample;
a data structure configured to store a set of genetic features comprising genetic features for each of a plurality of different organisms, and a plurality of reference genome sets each set comprising a plurality of diverse reference genomes for an organism;
a processor configured to: (i) identify a genotype of an organism in the sample by comparing the sequencing data to the set of genetic features, the set of genetic features comprising genetic features for each of a plurality of different organisms; (ii) select to which of the plurality of reference genome sets to compare the sequencing data; (iii) compare the sequencing data to the selected set of reference genomes; (iv) identify, based on the comparison, with which reference genome in the selected set of reference genomes the sequencing data most closely aligns, the identification further comprising an identification of a species or substrain of the organism in the sample; and
a user interface configured to report of one or more of the identified genotype of the organism in the sample and the identification of the species or substrain of the organism in the sample.

12. The system of claim 11, wherein the processor is further configured to generate a genetic feature set by: (i) extracting, from a plurality of sequenced genomes representing two or more varieties of an organism, one or more genetic features unique to the organism; (ii) normalizing the one or more extracted genetic features; and (iii) storing the normalized set of genetic features in memory.

13. The system of claim 11, wherein the processor is further configured to generate each of the plurality of reference genome sets by: (i) selecting a plurality of reference genomes for an organism; (ii) identifying, using the set of genetic features for that organism, the sequence similarity of each of the selected plurality of reference genomes; (iii) curating the plurality of reference genomes based on the identified sequence similarity; and (iv) storing the curated reference genome set in memory.

14. The system of claim 11, wherein the processor is further configured to: determine, based on the identified genotype of the organism, that the sequencing data comprises sequences from two or more organisms; and separate the sequencing data from each of the two or more organisms in the sample into a separate sequencing data file for each organism.

15. The system of claim 11, wherein the identification of the species or substrain of the organism in the report comprises a quantitative measure of the identification.
